(19) United States
(12) Patent Application Publication
(10) Pub. No.: US 2007/0276486 A1
(43) Pub. Date: Nov. 29, 2007

(54) Coated tracheostomy tube and stoma stent or cannula

(75) Inventors: Lewis H. Marten, Westwood, MA (US); Dennis Creedon, Sandwich, MA (US)

Correspondence Address: FROMMER LAWRENCE & HAUG, 745 FIFTH AVENUE- 10TH FL., NEW YORK, NY, 10151, US

(73) Assignee: E. Benson Hood Laboratories

(21) Appl. No.: 11/440,905

(22) Filed: May 25, 2006

(51) Int. Cl. A61F 2/20; A61M 16/04

(52) U.S. Cl. 623/9; 128/207.14

(57) ABSTRACT

The present invention is directed to an airway management device including a tube with a lumen extending therethrough and with the tube having an inner and outer surface. The outer surface and inner surface of the tube have a protective polymeric coating.

### BACKGROUND OF THE INVENTION

1. Field of the Invention

The present invention relates to devices used in the management of bodily airways. Specifically, this invention is directed to ways of preventing the accumulation of mucus, crusting and granulation on or around airway management devices.

2. Description of the Prior Art

A wide variety of airway management devices exist. Airway management devices may be used for a variety of reasons including the facilitation of speaking and breathing following a laryngectomy, the promotion of healing in the patient, the provision of an access point for forced ventilation of a patient and a variety of other uses including supplying oxygen to augment normal breathing. In particular, attention is directed to tracheostomy tubes and devices used in conjunction with a tracheostomy tube, for example, stoma stents, tracheal T-tubes, and transtracheal oxygen stents, among others. Those of skill in the art will appreciate that the management of bodily airways is not limited to those devices enabling respiration, but rather may extend to the panoply of devices relating to diseases of the larynx or pharynx.

A laryngectomy is one procedure that implicates airway management devices. A patient may undergo a laryngectomy in response to cancer of the larynx or possibly because of trauma to the region. A total laryngectomy will have profound effects on the patient. In a total laryngectomy, the larynx is surgically separated from the mouth, nose and esophagus and the entire larynx, including the vocal chords, is removed. The patient must thereafter use a laryngectomy tube for breathing. Further, due to the separation and lack of vocal chords, a patient may initially be unable to speak.

Some airway management devices are non-respiratory in the sense that they do not enable breathing directly, but still are related the respiratory system generally. In healthy individuals, the larynx is instrumental for speech, but for laryngectomicized individuals speech is still possible through alternative methods using speech prosthetic devices. Voice button devices, such as a “Panje” voice button and a “Groningen” voice button, help restore speech by allowing air, but not fluids, through an artificial fistula formed between the larynx and the esophagus.

Other non-respiratory airway management devices include salivary bypass tubes and esophageal tubes. Laryngectomies may create salivary fistulas which are problematic if formed over the laryngectomy stoma. This detrimental post-laryngectomy effect can be treated by using a salivary bypass tube. Also, following a laryngoesophagectomy, an esophageal tube may be used to bridge the gap between the pharyngostome and esophagostome.

Another example of a condition that may necessitate an airway management device is laryngeal stenosis. Laryngeal stenosis may occur if a patient has been intubated for a prolonged period of time. One device used in its treatment is a laryngeal umbrella keel. Laryngeal umbrella keels are sometimes used before removing a laryngeal stent, to insure reformation of a sharp anterior commissure and to prevent formation of an anterior web.

However, all of these devices suffer from several drawbacks. Airway management devices are often plagued by granulation, crusting and mucus build up. Further, such devices run the risk of compromising bodily walls and devices for airway management can be difficult for the patient to clean and maintain. In addition, ease of insertion and removal of complementary devices such as tubes can be hampered by the build up or encrustation of bodily fluids or by device fit friction. At the same time, however, another problem with airway management devices is the possibility of becoming accidentally dislodged. Thus there is a need for airway management devices which prevent build up of mucus, encrustation, or bodily fluids, yet remain firmly implanted in the patient with little likelihood of becoming accidentally dislodged. The present invention is directed towards a device solving these and other problems associated with the known devices.

### SUMMARY OF THE INVENTION

One aspect of the present invention is directed to an airway management device including a tube with a lumen extending therethrough and with the tube having an inner and outer surface. The outer surface and inner surface of the tube have a protective polymeric coating.

Another aspect of the present invention is directed to a stoma stent system including a tube with a lumen extending therethrough, an exterior flange formed on the proximal end of the tube, and a tracheal flange formed on the distal end of the tube. The tube fluidly connects the exterior flange to the tracheal flange. The exterior flange and the tracheal flange have a protective polymeric coating.

A still further aspect of the present invention is directed to a voice prosthesis device including a voice button with a lumen extending therethrough, a tracheal flange attached to a tracheal end of the voice button and an esophageal flange attached to the esophageal end of the voice button. The inner surface of the voice button, the tracheal flange, and the esophageal flange are coated with a protective polymeric coating.

The various features of novelty which characterize the invention are pointed out in particularity in the claims annexed to and forming a part of this disclosure. For a better understanding of the invention, its operating advantages and specific objects attained by its uses, reference is made to the accompanying descriptive matter in which preferred embodiments of the invention are illustrated.

### BRIEF DESCRIPTION OF THE DRAWINGS

For a more complete understanding of the invention, reference is made to the following description and accompanying drawings, in which:

FIG. 1 is a side view of a tracheostomy tube according to one aspect of the present invention;

FIG. 2 is a cut away view of a tracheostomy tube according to one aspect of the present invention;

FIG. 3 is a front view of a tracheostomy tube according to one aspect of the present invention;

FIG. 4 is a front view of a stoma stent according to one aspect of the present invention;

FIG. 5 is a cut away view of a stoma stent according to one aspect of the present invention;

FIG. 6 is a side view of a stoma stent according to one aspect of the present invention;

FIG. 7 is a side view of a T-tube according to one aspect of the present invention;

FIG. 8 is a cut away view of a T-tube according to one aspect of the present invention;

FIG. 9 is a front view of a T-tube according to one aspect of the present invention;

FIG. 10 is a front view of a transtracheal oxygen stent according to one aspect of the present invention;

FIG. 11 is a top view of a transtracheal oxygen stent according to one aspect of the present invention;

FIG. 12 is a cut away side view of a transtracheal oxygen stent according to one aspect of the present invention;

FIG. 13 is a side view of an oxygen delivery system for the transtracheal oxygen stent according to one aspect of the present invention;

FIG. 14 is a side view of a laryngectomy tube according to one aspect of the present invention;

FIG. 15 is a cut away view of a laryngectomy tube according to one aspect of the present invention;

FIG. 16 is a front view of a laryngectomy tube according to one aspect of the present invention;

FIG. 17 is a side view of a Panje voice button according to one aspect of the present invention;

FIG. 18 is a top view of a Panje voice button according to one aspect of the present invention;

FIG. 19 is a cut away view of a Panje voice button according to one aspect of the present invention;

FIG. 20 is a side view of a Groningen voice button according to one aspect of the present invention;

FIG. 21 is a cut away view of a Groningen voice button according to one aspect of the present invention;

FIG. 22 is a top view of a Groningen voice button according to one aspect of the present invention;

FIG. 23 is a side view of a salivary bypass tube according to one aspect of the present invention;

FIG. 24 is a cut away view of a salivary bypass tube according to one aspect of the present invention;

FIG. 25 is an end view of a salivary bypass tube according to one aspect of the present invention;

FIG. 26 is a front view of a laryngeal umbrella keel according to one aspect of the present invention;

FIG. 27 is a side view of a laryngeal umbrella keel according to one aspect of the present invention;

FIG. 28 is a top view of a laryngeal umbrella keel according to one aspect of the present invention;

### DETAILED DESCRIPTION

The present invention overcomes the shortcomings of the prior art by coating airway management devices with a protective polymeric coating. One suitable type of polymer is parylene. Parylene is the name for a series of polymers based on the monomer, para-xylene (p-xylene), or 1,4 dimethyl-benzene. There are three commercially available variations of parylene that display differences at the monomeric level: parylene N, parylene C, and parylene D. In one preferred embodiment, the instant invention uses parylene N or parylene C. Parylene is applied in a thickness of about 0.00003″ to 0.0001″ and more preferably in a thickness of about 0.00005″.

The backbone of the parylene polymer is made entirely of carbon and thereby is not vulnerable to hydrolytic breakdown in an aqueous environment. Parylene also has excellent properties as a film lubricant and its coefficient of friction approaches TEFLON®. Also, with a dielectric constant relatively independent of frequency and temperature, parylene also is an excellent electric insulator.

The devices contemplated by the present invention can be formed using any number of materials conventional to those skilled in the art for airway management devices. For example, one preferable material is medical grade silicone but other materials such as polyvinylchloride (PVC) could also be used without departing from the scope of the present invention.

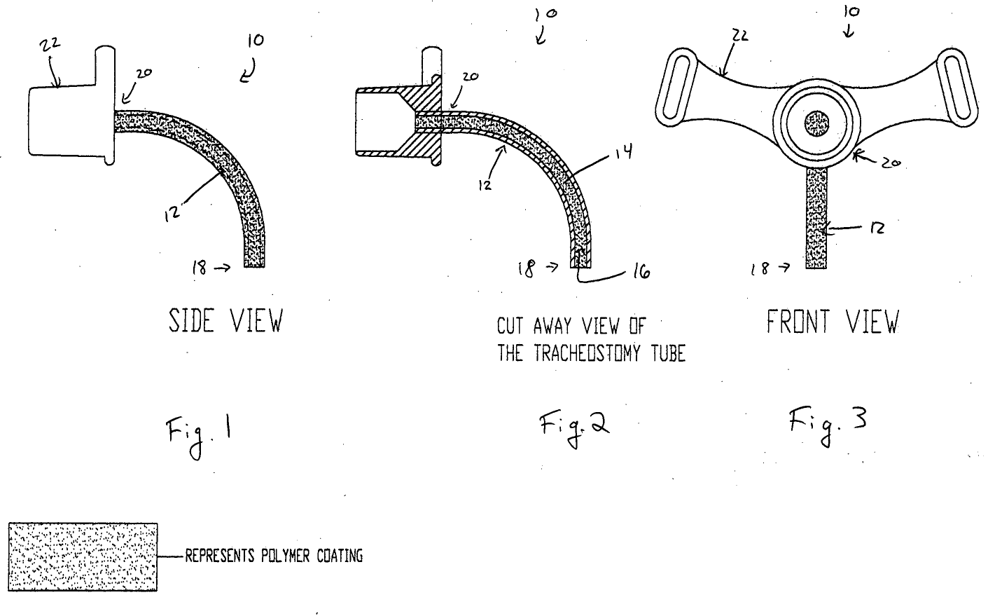

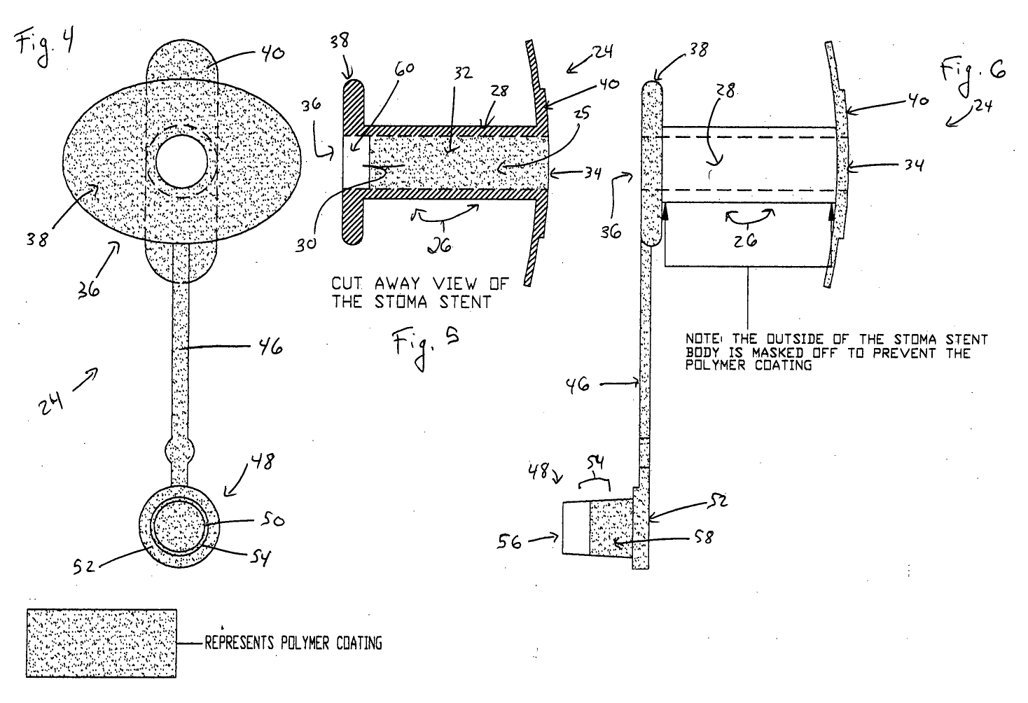

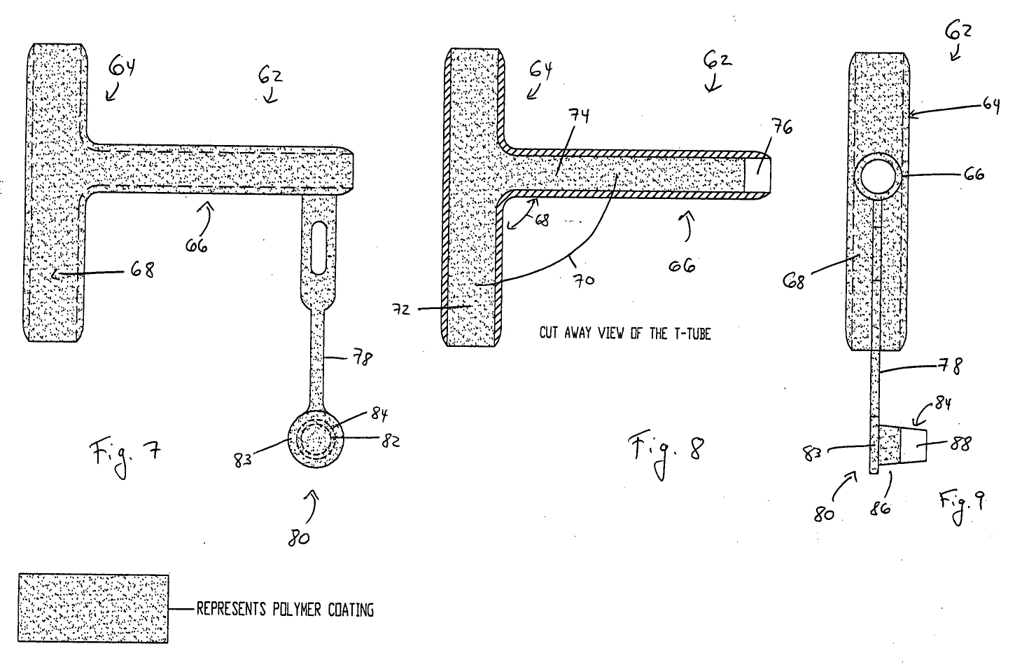

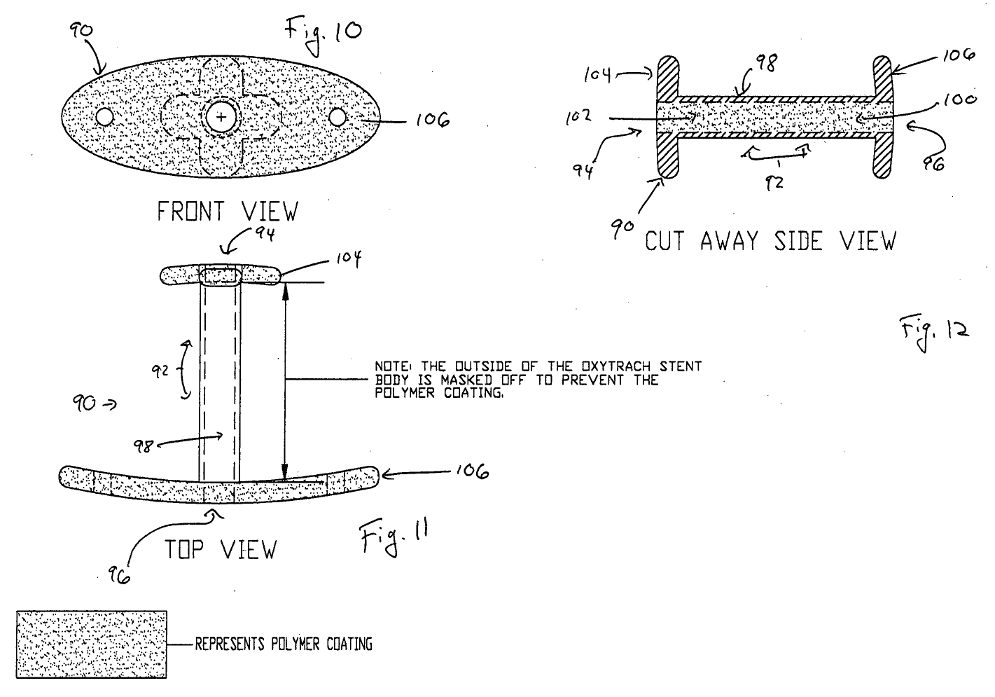

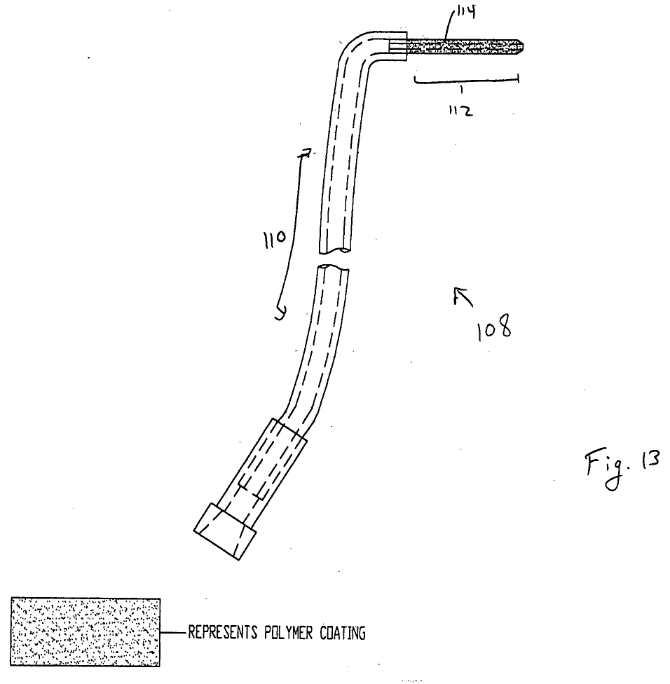

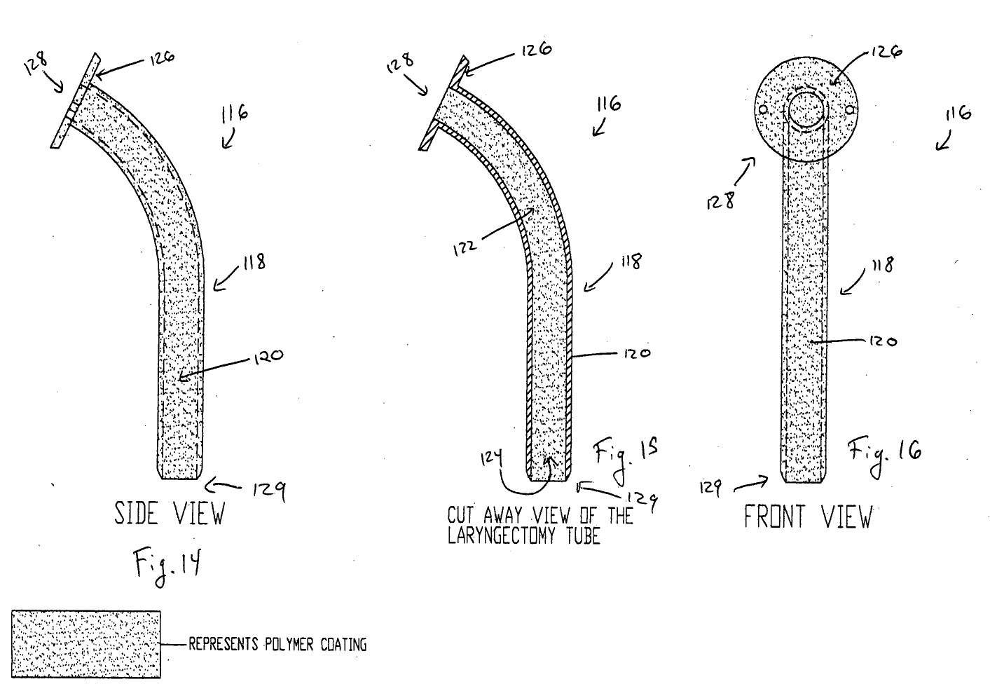

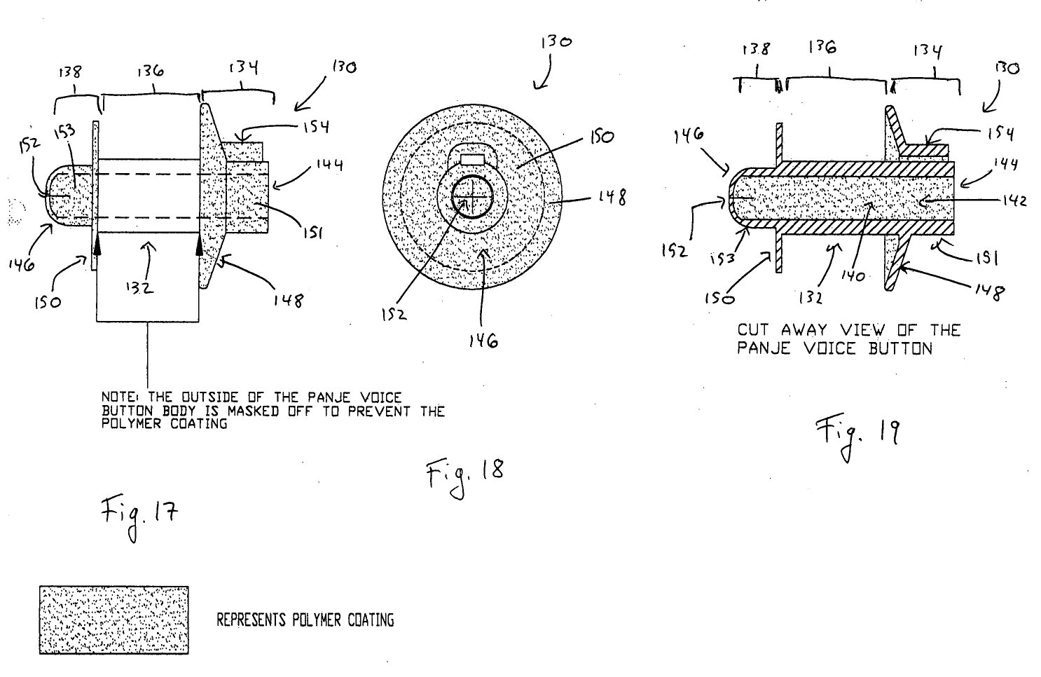

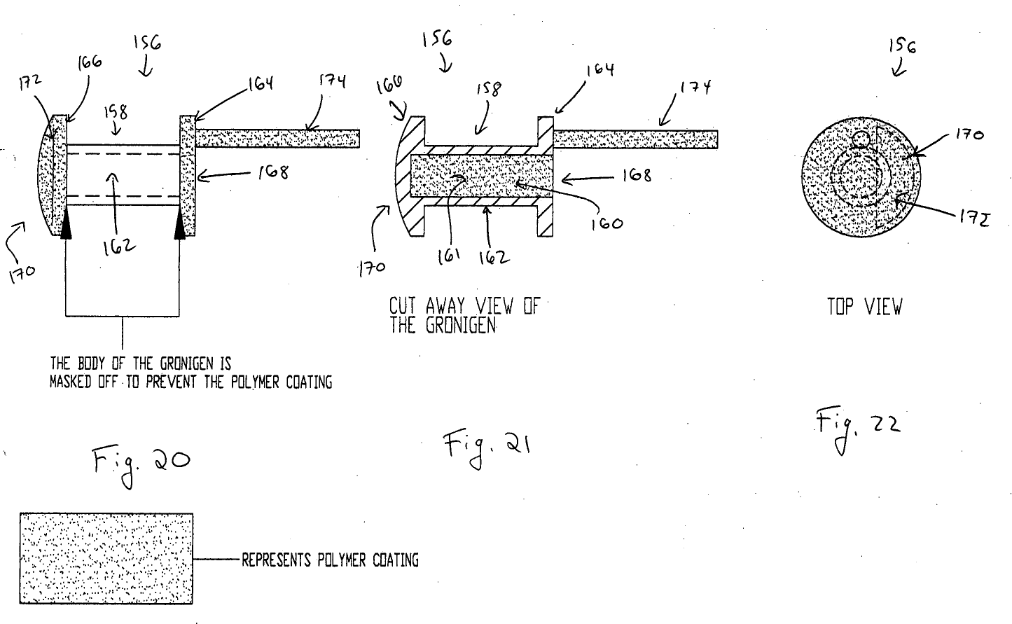

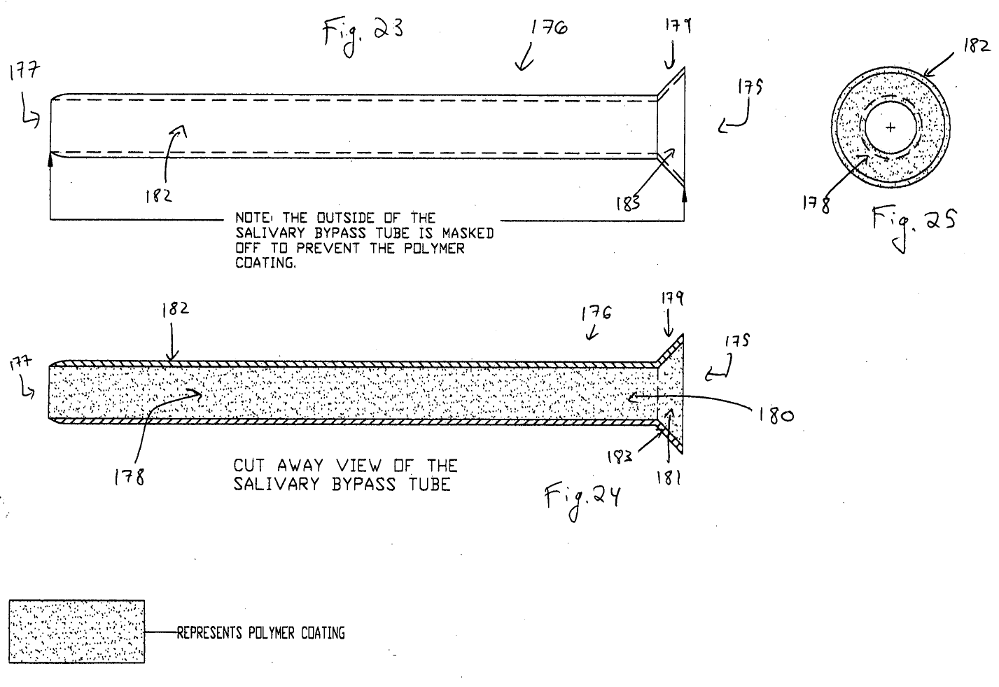

One embodiment of the present invention is a protectively coated tracheostomy tube as shown in FIGS. 1-3. Areas with a protective polymeric coating are illustrated by the shaded regions. FIG. 1 is a side view of a tracheostomy tube 10. The tube 10 is open at the distal end 18 and the proximal end 20. Tube 10 has an outer surface 12 and a lumen 16 extending therethrough having an inner surface 14. A neck plate 22 is attached at the proximal end 20, which in use rests against a patient's neck. One embodiment of the invention contemplates coating both the outer surface 12 of the tube 10 with a protective polymeric coating, as depicted in FIGS. 1 and 3, as well as and the inner surface 14 of the lumen 16, as depicted in FIG. 2. Coating the tracheostomy tube 10 in this manner serves to control mucus and granulation accumulation both internally and externally. In addition, such coating serves to ease in the insertion and removal of suction tubes. The neck plate 22 is preferably not coated with the polymeric protective coating. Those of skill in the art will appreciate that a cannula may be inserted to the lumen 16 of the tube 10. As with the tube 10, the cannula (not shown) may be coated internally and externally with a polymeric protective coating to ease insertion and prevent mucus build up or granulation.

FIGS. 4-6 show a stoma stent 24 in accordance with another embodiment of the present invention. Surrounding the proximal end 36 of the stoma stent 24 is an exterior flange 38 and surrounding the distal end 34 is a tracheal flange 40. The exterior flange 38 and the tracheal flange 40 are fluidly connected by a tube 26 with an outer surface 28 and with a lumen 25 extending therethrough and having an inner surface 30. The inner surface 30 has a portion 32 proximal to the tracheal flange 40 and a portion 60 proximal to the exterior flange 38.

The exterior flange 38 and the tracheal flange 40 are both coated with a protective coating. The outer surface 28 is not coated with a protective coating in order to prevent the stoma stent 24 from being coughed out of position. The inner surface 30 of the lumen 25 is coated with a protective polymeric material except for the portion 60 proximal to the exterior flange 38. The distal portion 60 is not coated. Coating the tracheal flange 40 with protective material prevents the accumulation of granulation, crusting and mucus. Further, the smoother surface reduces the possibility of compromising the tracheal wall of the patient. With regard to the exterior flange 38, the protective coating prevents the accumulation of granulation, crusting and mucus and allows for easier cleaning and maintenance of the stoma stent 24. The uncoated portion 60 provides enhanced friction to hold an item such as a cannula or a plug 48 firmly in the stoma stent.

As seen in FIGS. 4 and 6, also attached to the exterior flange 38 is a tether 46 connected to a plug 48. The plug 48 has a cylindrical inner wall 50 and a tapered outer wall 54 attached axially to a base 52, as shown in FIGS. 4 and 6. The tapered outer wall 54 has a portion 58 proximal to the base 52 and a portion 56 distal to the base 52. The distal portion 56 is not coated. The plug 48 is of a dimension and configuration to fit tightly within the lumen 30 of the tube 26 with base 52 abutting the exterior flange 38 and the uncoated portion 56 of the plug 48 secured to the uncoated portion 60 of the stoma stent 24.

Another embodiment of the invention is a polymer coated tracheal T-tube 62, as shown in FIGS. 7-9. FIG. 7 is a side view of a tracheal T-tube with a vertical arm 64 and a horizontal arm 66 arranged perpendicularly to the vertical arm 64 in a T shape. Joining the tube arms 64 and 66 forms a T-tube 62 having a single lumen 70 and one outer surface 68. Inner surface 72 is located within the vertical arm 64. Inner surface 74 is located within the horizontal arm 66. Inner surface 76 also is located within the horizontal arm 66 at a location distal to the vertical arm 64. The outer surface 68 of the T-tube 62 is coated with a protective material. Further, the inner surfaces 72 and 74 of the horizontal and vertical arms 66 and 64 are coated with a protective layer which allows for ease of insertion/removal of suction catheter, if necessary. However, the inner surface 76 of the horizontal arm 66 is not coated with a protective coating.

As shown by FIGS. 7 and 9, a tether 78 is attached to the horizontal arm 66 and connects a plug 80 to the horizontal arm 66. The plug 80 has a cylindrical wall 82 attached axially to a base 83 with a tapered outer wall 84, as shown in FIGS. 7 and 9. The tapered outer wall 84 has a proximal portion 86 and a distal portion 88 to the base 83. The plug 80 is of a dimension and configuration to fit tightly adjacent to the inner surface of the distal portion 76 of the horizontal arm 66 of the T-tube 62. Correspondingly, the distal portion 88 of the outer wall 84 of plug 80 is not coated ensuring a secure fit.

In yet another embodiment, FIGS. 10-12 show a transtracheal oxygen stent 90 coated with protective polymer. FIG. 11 shows a top view of a transtracheal oxygen stent 90 having a tube 92 with a lumen 102 extending therethrough having an inner surface 100. The tube 92, having an outer surface 98, is open at the proximal end 94 and the distal end 96. A tracheal flange 106 is attached at the distal end 96 of the tube 92 and an exterior flange 104 is attached at the proximal end 94, as shown in FIGS. 11 and 12. The tracheal flange 106 and the exterior flange 104 are both coated with a protective coating. Coating the tracheal flange 106 with protective material prevents the accumulation of granulation, crusting and mucus. Further, the smoother surface reduces the possibility of compromising the tracheal wall. With regard to the exterior flange 104, the protective coating prevents the accumulation of granulation, crusting and mucus and allows for easier cleaning and maintenance. What is more, as shown by FIG. 12, the inner surface 100 of the lumen 102 is coated with a protective coating. However, as shown by FIG. 11, the external surface 98 of the tube 92 does not have a coating. Coating the inner surface 100 with a protective material will prevent accumulation of crusting and mucus. The external surface 98 is not coated with a protective coating to prevent the stent 92 from being coughed out of the stoma formed in the patient.

In a further embodiment, the transtracheal oxygen stent 92, as shown in FIGS. 10-12 is adaptable for use with the oxygen delivery catheter 108 shown in FIG. 13 and is comprised of tubing 110 that connects the stent shown in FIGS. 19-21 to an oxygen source (not shown). The tubing 110 has a connecting portion 112 with an outer surface 114 that fits within the lumen 102 of the transtracheal oxygen stent 92, as shown in FIGS. 10-12. The outer surface 114 of the connecting portion 112 has a protective polymeric coating to ease insertion.

FIGS. 14-16 show another embodiment of the present invention, namely a laryngectomy tube 116 coated with a protective polymer. The laryngectomy tube 116 comprises a curved tube 118 with an outer surface 120. The tube 118 has a proximal end 128, a distal end 129 and a lumen 124 which extends therethrough having an inner surface 122. A neck plate 126 is attached at the proximal end 128 of the tube 118. The neck plate 126, outer surface 120 and inner surface 122 are all coated with the protective polymer.

Another embodiment of the invention is a protectively coated device for voice prosthesis. In one embodiment, shown in FIGS. 17-19, a voice button 130 has three segments: 134, 136, and 138, and a lumen 142 extending therethrough. The voice button 130 has an open tracheal end 144 and a valved 152 esophageal end 146. The entire inner surface 140 of the lumen 142 is protectively coated to prevent crusting and mucus. The first segment is the tracheal segment 134. The tracheal segment 134 includes a tracheal flange 148. The external surface 151 including the surface of the tracheal flange 148 of the tracheal segment 134 has a protective coating. Coating the tracheal flange 148 prevents granulation, crusting and mucus in the device as well as allowing for easier cleaning and maintenance. The second segment is the intrafistular segment 136. In use this segment rests in the fistula between the trachea and the esophagus. The lumen 142 extends between the tracheal flange 148 and the esophageal flange 150 and through the intrafistular segment 136. The external surface 132 of the intrafistular segment 136 is not protectively coated. The third segment is the esophageal segment 138, including the esophageal flange 150 to the esophageal end 146, which contains a valve system 152 that allows air to pass through the voice button 130 from the lungs but prevents fluids from the esophagus from passing in the opposite direction. The external surface 153 of the esophageal segment 138 is protectively coated. Similarly, the esophageal flange 150 has a protective coating to prevent granulation, crusting, and mucus accumulation. Coating the esophageal flange 150 will also prevent the accumulation of food particulates. Another embodiment may include a string 154 attached to the tracheal segment 134 that may be used to retrieve the device from the fistula. This string 154 may also be coated with a protective polymeric coating. One skilled in the art would know the device previously described as a “Panje voice button.”

Another embodiment of the invention is a protectively coated voice button 158, as shown in FIGS. 20-22. In this embodiment, the voice button 158 has a lumen 160 extending therethrough having an inner surface 161. The voice button 158 has an open tracheal end 168 and an esophageal end 170 with a valve 172. The tracheal end 168 includes a tracheal flange 164 and the esophageal end 170 includes an esophageal flange 166. The entire inner surface 161 of the lumen 160 is coated by a protective coating, however, the external surface 162 of the voice button 158 is not coated. The tracheal flange 164 is coated with a protective coating as is the esophageal flange 166. The valve 172 allows air to pass through the voice button 158 from the lungs but prevents fluids from the esophagus to pass in the opposite direction. Another embodiment includes a string 174 attached to the laryngeal flange 164 to retrieve the device 156 from the fistula. In one embodiment, the string 174 is also coated with a protective coating. One skilled in the art would know the device previously described as a “Groningen voice button.”

Yet another embodiment the present invention is a non-respiratory airway management device such as the salivary bypass tube 176 shown in FIG. 23-25. According to the present invention, the salivary bypass tube 176 has an exterior surface 182 and a lumen 180 which extends therethrough. The inner surface 178 of the lumen 180 is coated with a protective polymeric coating. However, the exterior surface 182 of the tube 176 is not coated with a protective polymeric coating. In one embodiment of the invention, the tube 176, having proximal 175 and distal 177 ends, further comprises a funnel 179 attached to the proximal end 175. The inner surface 181 of the funnel 179 has a protective polymeric coating but the outer surface 183 of the funnel 179 is not so coated.

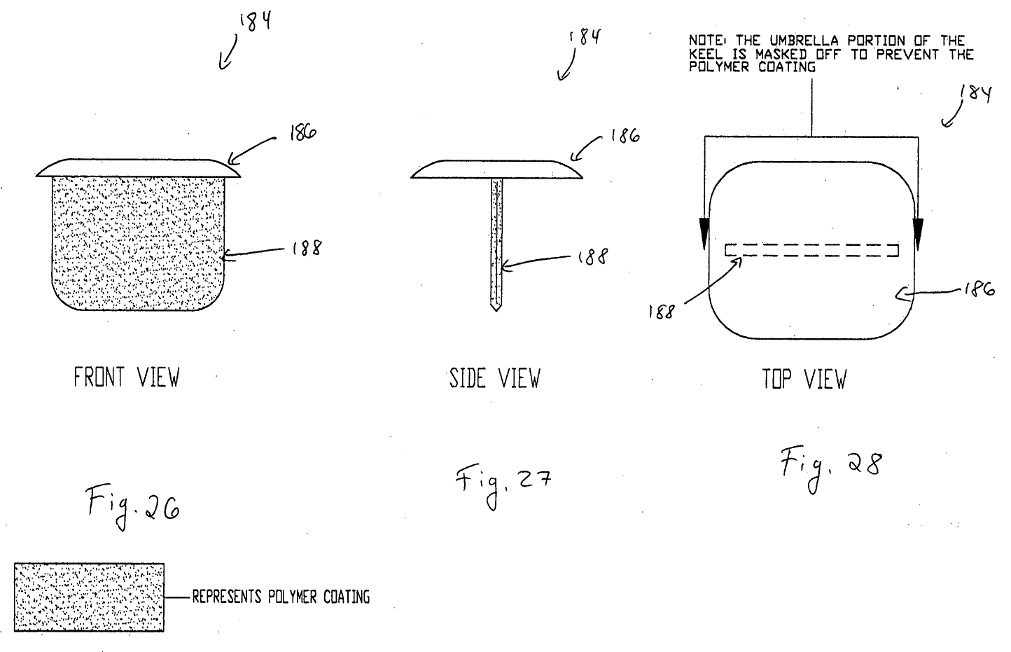

A further embodiment of the present invention is a non-respiratory airway management device such as the laryngeal umbrella keel 184 shown in FIGS. 26-28. The laryngeal umbrella keel 184 is comprised of an umbrella-like extralaryngeal cover 186 and a thin intralaryngeal insert 188, joined perpendicularly in a T-shape. The umbrella-like extralaryngeal cover 186 is not coated with a protective polymer. The thin intralaryngeal insert 188 is protectively coated. Coating the thin intralaryngeal cover 188 prevents the formation of granulation and eases removal of the device, thereby reducing the possibilities of vocal chord adhesions.

While the present invention has been particularly shown and described in conjunction with preferred embodiments thereof, it will be readily appreciated by those of ordinary skill in the art that various changes may be made without departing from the spirit and scope of the invention. Therefore, it is intended that the appended claims be interpreted as including the embodiments described herein as well as all equivalents thereto.

1. An airway management device comprising:
a tube with a lumen extending therethrough, said tube having an inner surface and an outer surface,
wherein said outer surface of said tube and said inner surface of said lumen have a protective polymeric coating.

2. The airway management device of claim 1, wherein said protective polymeric coating is parylene.

3. The airway management device of claim 1, wherein said tube is a tracheostomy tube.

4. The airway management device of claim 3, further comprising a cannula.

5. The airway management device of claim 4, wherein said cannula has a protective polymeric coating.

6. The airway management device of claim 5, wherein said protective polymeric coating is parylene.

7. The airway management device of claim 1, further comprising a neck plate attached at the proximal end of said tube.

8. The airway management device of claim 1, wherein said tube is a laryngectomy tube.

9. The airway management system of claim 8, further comprising a neck plate attached at the proximal end of said tube.

10. The airway management device of claim 9, wherein said neck plate has a protective polymeric coating.

11. The airway management device of claim 10, wherein said protective polymeric coating is parylene.

12. The airway management device of claim 1, wherein only a portion of said inner surface of said lumen has a protective polymeric coating.

13. The airway management device of claim 12, wherein said tube has a vertical arm and a horizontal arm joined perpendicularly to said vertical arm, and has a continuous outer surface and a single lumen.

14. The airway management device of claim 13, further comprising a tether and a plug, wherein said tether is attached to the proximal end of said horizontal arm and said plug is attached at its base to said tether.

15. The airway management device of claim 14, wherein said tether is coated with a protective polymeric coating.

16. The airway management device of claim 15, wherein said protective polymeric coating is parylene.

17. The airway management device of claim 14, wherein a portion of the outer wall of said plug is coated with a protective polymeric coating.

18. The airway management device of claim 17, wherein the proximal portion of said outer wall to said base is coated with a protective polymeric coating.

19. The airway management device of claim 18, wherein said protective polymeric coating is parylene.

20. The airway management device of claim 13, wherein the inner surface of said lumen within said vertical arm and the inner surface of said lumen within said horizontal arm proximal to said vertical arm have a protective polymeric coating.

21. The airway management device of claim 20, wherein said protective polymeric coating is parylene.

22. The airway management device of claim 14, wherein said device is a tracheal T-tube.

23. A stoma stent system comprising:
a tube with a lumen extending therethrough;
an exterior flange formed on the proximal end of said tube;
a tracheal flange formed on the distal end of said tube; said tube fluidly connecting said exterior flange to said tracheal flange; and
wherein said exterior flange and tracheal flange have a protective polymeric coating.

24. The stoma stent system of claim 23, wherein the protective polymeric coating is parylene.

25. The stoma stent system of claim 23, wherein the inner surface of said lumen is coated with a protective polymeric coating.

26. The stoma stent system of claim 25, wherein the protective polymeric coating is parylene.

27. The stoma stent system of claim 25, wherein the system is a transtracheal oxygen stent system.

28. The stoma stent system of claim 27, further comprising an oxygen delivery system having a connecting portion that fits within said lumen of said transtracheal oxygen stent and wherein the outer surface of said connecting portion has a protective polymeric coating.

29. The stoma stent system of claim 28, wherein said protective polymeric coating is comprised of parylene.

30. The stoma stent system of claim 25, wherein the inner surface of said lumen is only partially coated with a protective polymeric coating.

31. The stoma stent system of claim 30, wherein the protective polymeric coating is parylene.

32. The stoma stent system of claim 30, wherein the coated portion of said inner surface is the proximal portion to said tracheal flange.

33. The stoma stent system of claim 32, wherein said protective polymeric coating is parylene.

34. The stoma stent system of claim 25, further comprising a tether and a plug, wherein said tether is attached to said exterior flange and said plug is attached to said tether.

35. The stoma stent system of claim 34, wherein said tether is attached to the base of said plug.

36. The stoma stent system of claim 35, wherein said tether is coated with a protective polymeric coating.

37. The stoma stent system of claim 36, wherein said protective polymeric coating is parylene.

38. The stoma stent system of claim 34, wherein only a portion of said plug is coated with a protective polymeric coating.

39. The stoma stent system of claim 38, wherein said protective polymeric coating is parylene.

40. The stoma stent system of claim 38, wherein the proximal portion of said plug to said tether is coated with a protective polymeric coating.

41. The stoma stent system of claim 40, wherein said protective polymeric coating is parylene.

42. A voice prosthesis device comprising:
a voice button with a lumen extending therethrough;
a tracheal flange attached to a tracheal end of said voice button;
an esophageal flange attached to an esophageal end of said voice button; and
wherein the inner surface of said voice button, said tracheal flange, and said esophageal flange are coated with a protective polymeric coating.

43. The voice prosthesis device of claim 42, wherein said protective polymeric coating is parylene.

44. The voice prosthesis device of claim 42, further comprising a valve on said esophageal flange.

45. The voice prosthesis device of claim 42 further comprising a string wherein said string is coated with a protective polymeric coating.

46. The voice prosthesis device of claim 45, wherein said protective polymeric coating is parylene.

47. The voice prosthesis device of claim 42, wherein the outer surface of said voice button is comprised of a tracheal segment, a intrafistular segment, and an esophageal segment wherein said tracheal segment and said esophageal segment are coated with a protective polymeric coating.

48. The voice prosthesis device of claim 47, wherein said protective polymeric coating is parylene.

49. The voice prosthesis device of claim 47, wherein said device is a Panje voice button.

50. The voice prosthesis device of claim 47, further comprising a string, wherein said string is coated with a protective polymeric coating.

51. The voice prosthesis device of claim 50, wherein said protective polymeric coating is parylene.

52. The voice prosthesis device of claim 42, wherein said voice prosthesis device is a Groningen voice button.

53. An airway management device comprising at least two components:
a first component having a surface extending essentially perpendicular to a second component;
said second component extending perpendicularly away from said first component;
said second component having a second surface; and
wherein at least said second surface is coated with a protective polymeric coating.

54. The airway management device of claim 53, wherein said protective polymeric coating is parylene.

55. The airway management device of claim 53, wherein said first component is a funnel, said second component is a tube, and said airway management device is a salivary bypass tube.

56. The airway management device of claim 55, wherein said protective polymeric coating is parylene.

57. The airway management device of claim 55, wherein the inner surface of said tube is said second surface.

58. The airway management device of claim 55, wherein said funnel has a protective polymeric coating on the inner surface of said funnel.

59. The airway management device of claim 58, wherein said protective polymeric coating is parylene.

60. The airway management device of claim 53, wherein said first component is an umbrella-like extralaryngeal cover, said second component is a thin intralaryngeal insert and said airway management device is a laryngeal umbrella keel.

61. The airway management device of claim 60, wherein said protective polymeric coating is parylene.

62. The airway management device of claim 60, wherein said second surface is the entire surface of said thin intralaryngeal insert.

63. A cannula for use in an airway management device wherein said cannula has a protective polymeric coating.

64. The canulla of claim 63, wherein said protective polymeric coating is parylene.

65. The canulla of claim 63, wherein said protective coating is on both inside and outside of the canulla.

66. The canulla of claim 65, wherein said protective polymeric coating is parylene.
